(19) United States
(12) Patent Application Publication
(10) Pub. No.: US 2006/0121441 A1
(43) Pub. Date: Jun. 8, 2006

(54) Methods of determining the effect of an agent on diploid cells and/or on the pattern of expression of polypeptides expressed therewith

(76) Inventor: Micha Spira, Jerusalem (IL)

Correspondence Address: Martin Moynihan;Anthony Castorina, Suite 207, 2001 Jefferson David Highway, Arlington, VA, 22202, US

(21) Appl. No.: 10/528,785

(22) PCT Filed: Sep. 24, 2003
(86) PCT No.: PCT/IL03/00763

§ 371 Date: Oct. 17, 2005

(30) Foreign Application Priority Data
Sep. 24, 2002 (US) 60412840

(51) Int. Cl. C12Q 1/00; C12Q 1/68; C12N 5/08; C12N 15/87

(52) U.S. Cl. 435/4; 435/6; 435/455; 435/368

(57) ABSTRACT

A method of determining the effect of an agent on a diploid cell and/or on an expression or activity of a polypeptide expressed within the diploid cell is provided. The method is effected by: (a) administering an exogenous RNA molecule encoding the polypeptide into the diploid cell; (b) contacting the diploid cell with the agent; and (c) monitoring a phenotype of the diploid cell and/or the expression or activity of the polypeptide within the diploid cell, thereby determining the effect of the agent on the diploid cell and/or on the expression or activity of the polypeptide expressed within the diploid cell.

### FIELD AND BACKGROUND OF THE INVENTION

The present invention relates to methods of determining the effect of an agent on diploid cells and/or on the pattern of expression of polypeptides expressed therewith.

The extensive effort to sequence the human genome as well as the genome of other vertebrates and invertebrates is expected to revolutionize medicine and agriculture. To effectively use the libraries of gene sequences, the physiological or pathological functions of a given gene or groups of genes has to be understood. This can be achieved by the use of reliable platforms to express the genes and then examine the outcome of their actions at different levels. The physiological or pathological effects of a gene, or a group of genes, can be studied on the behavioral or morphological levels of a whole animal, system-tissue, cellular or biochemical levels.

Visualization of the spatiotemporal distribution of the gene's product and their relationship to other proteins, in real time and under different physiological and pathological contexts, is needed in order to analyze their mechanisms of action and their various functions. Accordingly, chimeric DNA constructs comprising reporter genes [encoding reporter proteins such as green fluorescent protein (GFP) and derivatives (EGFP, YFP, etc.), β-galactosidase, β-glucoronidase, etc.] have been increasingly used in biological research. The reporter genes are typically fused to the genes under study. The fusion constructs are introduced in to cells, expressed and visualized. Reporter genes have a wide variety of applications including visualization of the temporal and spatial distribution of genes expression products at the single cell level.

Yet, because of technical difficulties such as low transfection rate and poor spatial-temporal resolution, the use of reporter genes for on line visualization in differentiated neurons, is not used. Thus, functional and pathological interactions between gene products and the neuronal environment cannot be easily studied.

Difficulties in visualizing gene expression in neutonal cells, are well illustrated by studies conducted on cultured Aplysia neurons. These cells have been extensively used to study neuroplasticity (for review see Kandel 2001), regeneration after trauma (Spira et al., 1993, 1996, 1999, 2000), pharmacology, second messenger systems, neuronal development, synaptogenesis and neuronal network formation (see litrature cited by Kandel 2001) thereby providing cellular, molecular, biochemical, pharmacological and biophysical background information that can be utilized for studying gene functions.

Prior art studies have demonstrated that because of the large size of these neurons, localized intracellular changes can be directly visualized (Ziv and Spira 1997, 1998; Gabso et al., 1997; Gitler and Spira 1998, 2002). However, all prior attempts to transform Aplysia via DNA microinjection, including DNA encoding GFP, resulted in poor gene expression (Chang et al., 2000; DesGroseillers et al., 1987, Kaang et al., 1992; 1993; Kaang 1996a,b; Kim and Kaang 1998; Lee et al., 2000; Martin et al., 1995; Zhao et al., 1994). Therefore, DNA constructs encoding reporter genes such as GFP, have not been useful for studying gene expression in Aplysia.

Mochida et al (1990), illustrated that injection of mRNA coding for tetanus and botulinum toxins into Aplysia neurons led to down regulation in neurotransmitter release. However, while the pathological effect of (extremely low concentration) butulinum and tetanus toxins was detectable (suppressing neurotransmitter release), their expression level was far too low for directly detecting, or visualizing, gene expression products in situ.

While reducing the present invention to practice, the present inventors devised a novel approach which traverses the limitations inherent to prior art methods of studying expression of gene products in differentiated cells, and in neurons in particular. Thus, the present invention provides a useful, convenient, rapid and cost effective tool for directly visualizing fate and function of gene expression products in differentiated cells.

### SUMMARY OF THE INVENTION

According to one aspect of the present invention there is provided A method of determining the effect of an agent on a diploid cell and/or on an expression or activity of a polypeptide expressed within the diploid cell, the method comprising: (a) administering an exogenous RNA molecule encoding the polypeptide into the diploid cell; (b) contacting the diploid cell with the agent; and (c) monitoring a phenotype of the diploid cell and/or the expression or activity of the polypeptide within the diploid cell, thereby determining the effect of the agent on the diploid cell and/or on the expression or activity of the polypeptide expressed within the diploid cell.

According to further features in preferred embodiments of the invention described below, the diploid cell is a differentiated cell.

According to still further features in the described preferred embodiments the diploid cell is a neuron.

According to still further features in the described preferred embodiments the administering is effected by microinjection.

According to still further features in the described preferred embodiments the exogenous RNA molecule is a capped messenger RNA.

According to still further features in the described preferred embodiments the polypeptide is conjugated to a detectable label selected from the group consisting of green fluorescent protein (GFP), derivatives of GFP, luciferase, β-glucoronidase, β-galactosidase, and chloramphenicol acetyltransferase.

According to still further features in the described preferred embodiments the monitoring is effected by:

(i) fluorescent microscopy;

(ii) protein expression assay; and/or

(iii) assaying enzymatic activity.

According to still further features in the described preferred embodiments the exogenous RNA molecule encoding the polypeptide is a chimeric RNA molecule including a first sequence region encoding the polypeptide and a second sequence region encoding a reporter molecule, wherein the first and the second sequence regions are linked via an internal ribosome entry site sequence.

According to still further features in the described preferred embodiments the exogenous RNA molecule encoding the polypeptide is a chimeric RNA molecule including a first sequence region encoding the polypeptide and a second sequence region encoding a reporter molecule, wherein the first and the second sequence regions are in-frame linked.

According to another aspect of the present invention there is provided a neuronal cell comprising a chimeric RNA molecule including a first sequence region encoding a polypeptide of interest and a second sequence region encoding a reporter molecule, wherein the first and the second sequence regions are linked via an internal ribosome entry site sequence.

The present invention successfully addresses the shortcomings of the presently known configurations by providing methods of determining the effect of an agent on diploid cells and/or on the pattern of expression polypeptides expressed therewith.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-d are photomicrographs depicting expression of EYFP in cultured Aplysia neurons. EYFP was injected into cultured Aplysia neurons 20 hours prior to imaging. FIGS. 1a and 1c are differential interference contrast images of the neuron. FIGS. 1c and 1d are enlargements of the neural segment within the rectangle of FIG. 1a. FIGS. 1b and 1d are confocal images. Note that the fluorescent signal is evenly distributed in the axoplasm. The confocal images were produced using the following setting: laser intensity 5%; iris setting 1.6; gain 100.

FIGS. 2a-b are photomicrographs depicting EGFP-actin bundles at the leading edge of a growth cone lamellipodium formed following axotomy. A B1 neuron was axotomyzed 28 hours following injection with mRNA encoding the fusion protein. Transection was followed by rapid extension of the growth cone lamellipodium. FIG. 2a is an image of the growth cone lamellipodium taken at the level of the glass substrate 29 min following axotomy. FIG. 2b is an image taken from the same region 3 mm above the substrate. Note that in FIG. 2a, actin puncta are seen along the axonal plasma membrane facing the substrate. The perimeters of the growth cone's lamellipodium contain radially oriented actin bundles. In FIG. 2b, 3 mm above the substrate, the actin polymerise along the axon's plasma membrane. The core of the axoplasm does not contain clear actin network.

FIGS. 3a-b are photomicrographs depicting the effect of Cytochalasin B on disassembly of actin bundles at the leading edge of growth cones. Shown are two growth cones formed by a cultured B1 neuron injected with mRNA encoding EGFP-actin fusion protein. FIG. 3a is a photomicrograph showing the leading edges of the growth cones containing bundles of EGFP labeled actin. FIG. 3b is a photomicrograph showing actin bundles disassembly and the formation of small actin aggregates within the collapsed growth cones and the axoplasm, nine minutes following bath application of 2 mM cytochalasin B.

FIGS. 4a-b are photomicrographs depicting the depolymerisation of microtubules within a growth cone formed following axotomy. A cultured B1 neuron was injected with mRNA encoding EGFP-tubulin fusion protein. FIG. 4a is a photomicrograph depicting EGFP labelled microtubules radiate from the growth cone centre towards the growth cone's lamellipodium perimeters. FIG. 4b is a photomicrograph depicting as in FIG. 4a, only twenty three minutes following bath application of 5 mM nocodosole the microtubules depolymerise.

FIGS. 5a-d are photomicrographs depicting alterations in the spatiotemporal distribution of EGFP-EB3 following axotomy. FIG. 5a is a photomicrograph depicting expression of EGFP-EB3 in the intact axon. FIG. 5b is a photomicrograph depicting expression of EGFP-EB3 20 seconds following axotomy. Note that a transient increase in the free intracellular calcium concentration was detected parallely (not shown). Further note the pattern of EGFP-EB3 “comet tails”-like fluorescent signal, associated with the plus end of the microtubules, dissipating from the tip of the transected axon. FIG. 5c is a photomicrograph depicting expression of EGFP-EB3 following the recovery of the free intracellular calcium concentration. Note that EGFP-EB3 reassociate with repolymerizing microtubules. FIG. 5d is a photomicrograph depicting expression of EGFP-EB3 10 minutes following axotomy. Note that the microtubules at the tip of the transected axon undergo additional changes that finally lead to the formation of vesicles trap surrounded by microtubules pointing their plus ends to a common center. The Golgi derived vesicles (not shown) were visualized by EGFP-SNAP 25.

### DESCRIPTION OF THE PREFERRED EMBODIMENTS

The present invention is of methods of determining the effect of an agent on diploid cells and/or on the pattern of expression of polypeptides expressed therewith. Specifically, the present invention can be used to identify agents which affect gene expression and function in neurons, such as the cultured neurons of Aplysia and thus serve as a system for modeling drug-gene interactions

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

In the post genomic era, an increased amount of genomic information has created new challenges for the biological research community and the pharmaceutical industry. These include, functional annotation of yet uncharacterized genes and efficient identification of target genes responsible for complex disease phenotypes and the use of such information for the development of new and specific classes of drugs.

One approach for obtaining such valuable information is by visualization of the spatial-temporal distribution of gene products, in real time and under different physiological and pathological conditions. Accordingly, chimeric DNA constructs including reporter genes such as fluorescent proteins, typically of the green fluorescent protein (GFP) family have been increasingly used.

Prior attempts to use such chimeric DNA constructs for real-time visualization of expression products in differentiated neurons were unsuccessful, due to very low levels of transcription in these cells and a limited pattern of expression [Kaang (1996a,b); Manseau (2001); Lee (2001)].

Injection of reporter molecules into differentiated neurons has also been attempted. For example, Mochida and co-workers injected mRNA of various toxins into cultured Aplysia neurons. As with intra-cell expression assays, expression products of micro-injected mRNA could not be detected visually, rather only functionally, indicating that only very low levels of expression could be achieved using this system [Mochida (1990)]. Other attempts to inject reporter mRNA (i.e., LacZ) into differentiated neuronal cells were unsuccessful [Kaang (1996a,b)].

While reducing the present invention to practice, the present inventor uncovered that direct microinjection of chimeric RNA molecules into diploid cells e.g., cultured Aplysia neurons, can be used to visualize gene expression in such cells and to study the effect of various agents on gene expression and cell fate.

As is illustrated in the Examples section which follows, microinjection of capped mRNA constructs of enhanced fluorescent protein linked to a given gene into the cytosol of cultured Aplysia neurons led, within hours of injection, to the translation and distribution of the fluorescentlly tagged protein in the differentiated neuron. This methodology was employed for the first time to demonstrate structural events which take place following axotomy of cultured Aplysia neurons.

Although mRNA injection of chimeric polypeptides into haploid cells (e.g., xenopus oocytes) is routinely practiced, it is well known that ploidy affects gene expression levels, and thus experiments demonstrating expression of injected chimeric mRNA in haploid cells cannot be reliably utilized to predict the outcome of such experiments in diploid cells [van Neck (1992) FEBS Lett. 297:189-195; Galitski (1999) Science 285:251-254].

Thus, the present invention provides a method of determining an effect of an agent on a diploid cell and/or on an expression or activity of a polypeptide expressed within the diploid cell.

As used herein the phrase “diploid cell” refers to a cell which has one chromosome from each parental set. The diploid cell according to this aspect of the present invention may be of a vertebrate (e.g., human) or invertebrate (e.g., Aplysia californica) animal origin. Preferably, the diploid cell of the present invention is a differentiated cell. According to a preferred embodiment of the present invention the differentiated cell is an Aplysia neuron (See the Background and Examples sections).

The method, according to the present invention is effected by administering an exogenous RNA molecule to the diploid cell.

As used herein the “exogenous RNA molecule” refers to an RNA molecule of naturally occurring nucleotides or analogues thereof which enhance stability and delivery of the exogenous RNA molecule. The exogenous RNA molecule of the present invention encodes an autologous or heterologous polypeptide, which localization, activity and/or level of expression are monitored to determine the effect of the agent thereon and/or on the diploid cell. Typically the exogenous RNA molecule is the mRNA product of in-vitro transcription of a DNA molecule as is further described hereinbelow.

Prior to, concomitant with or following introduction of the exogenous RNA molecule, the diploid cell is contacted with the agent. Finally, the phenotype of the diploid cell and/or expression or activity of the polypeptide is monitored to thereby determine the effect of the agent on the diploid cell and/or on the expression or activity of the polypeptide expressed within the diploid cell.

As mentioned hereinabove, the exogenous RNA molecule may be used to monitor the effect of the agent on the diploid cell (e.g., cell morphology).

Thus., the exogenous RNA molecule may encode a structural protein, such as actin, or a protein binding thereto (see Examples section which follows) or subcellular structure marker such as a cell surface protein which identifies the cell membrane.

Alternatively, the exogenous RNA molecule may encode a polypeptide of interest which activity or expression may be studied in response to treatment with the agent.

Preferably, the exogenous RNA molecule, according to this aspect of the present invention encodes a chimeric polypeptide which includes the polypeptide of interest fused in frame to a detectable polypeptide. It will be appreciated, however, that although the nature of the detectable polypeptide is of no significance, it should not alter the three dimensional structure of the polypeptide of interest in such fusions.

Alternatively, the chimeric mRNA can include an out-of-frame fusion of the two coding sequences encoding the polypeptide of interest and the detectable polypeptide provided that the downstream coding sequences is preceded by an internal ribosome entry site (IRES). IRES elements are able to bypass the ribosome scanning model of 5′ methylated Cap dependent translation and begin translation at internal sites [Pelletier and Sonenberg, (1988) Nature. 334(6180):320-5]. A number of IRES elements are known in the art such as, for example, the IRES elements of the picanovirus family (polio and encephalomyocarditis), which have been described by Pelletier and Sonenberg, (1988) supra, as well an IRES from a mammalian message [Macejak and Sarnow, (1991) Nature. 353(6339):90-4]. When the IRES element is present on an mRNA downstream of a translational stop codon, it directs ribosomal re-entry [Ghattas et al (1991) Mol. Cell. Biol. 11:5848-5959], which permits initiation of translation at the start of a second open reading frame). In this manner, multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

A number of polycistronic vectors are known in the art, which may be used in the present invention [see de Felipe (2002) Curr. Gene Ther. 2(3):355-78; Vagner (2001) EMBO Rep. 2(10):893-8]. For example, the bicistronic expression plasmid, pIRES1neo which is available from Clontech, Palo Alto, Calif. contains the human cytomegalovirus (CMV) major immediate early protein/enhancer followed by a multiple cloning site (MCS); a synthetic intron; and the encephalomyocarditis virus internal ribosome entry site (IRES), followed by the neomycin phosphotransferase gene, with a downstream bovine growth hormone polyadenylation signal.

As used herein the phrase detectable polypeptide refers to a polypeptide, which can be detected directly or indirectly. For example, the detectable polypeptide can be a fluorescer such as the polypeptides belonging to the green fluorescent protein family including the green fluorescent protein (GFP), the yellow fluorescent protein (YFP), the cyan fluorescent protein (CFP) and the red fluorescent protein (RFP) as well as their enhanced derivatives. In such a case, the detectable polypeptide can be detected via its fluorescence, which is generated upon the application of a suitable excitatory light. The detectable polypeptide can also be an enzyme which when in the presence of a suitable substrate generates chromogenic products. Such enzymes include but are not limited to alkaline phosphatase, β-galactosidase, β-D-glucoronidase (GUS) and the like. Alternatively, the detectable polypeptide can be an epitope tag, a fairly unique polypeptide sequence to which a specific antibody can bind without substantially cross reacting with other cellular epitopes. Such epitope tags include a Myc tag, a Flag tag, a His tag, a Leucine tag, an IgG tag, a streptavidin tag and the like. Further detail of polypeptide labels can be found in Misawa et al.

A number of methods for generating and purifying RNA molecules are known in the art and described in the Materials and Experimental Procedures section of the Examples section, which follows and in Sambrook, Fritsch, Maniatis (1989). Molecular Cloning: A laboratory manual. CSH Laboratory Press

For example the exogenous RNA moclecules of the present invention may be generated by in vitro transcription. In vitro transcription is the process by which RNA polymerase, in the presence of purified reaction components, mimics in vivo transcription and directs the generation of an RNA transcript from a DNA template.

Thus, a DNA polynucleotide encoding the exogenous RNA molecule of the present invention is ligated into a nucleic acid vector. A number of vectors designed for in-vitro transcription as well as cloning purposes are known in the art and may be commercially obtained (see www.promega.com/vectors/).

It will be appreciated that high quality DNA is required in order to achieve high yield (i.e., above 0.7 mg/ml) of in-vitro generated RNA transcripts. Template DNA may be linear or circular, including supercoiled. Measures are taken that the supercoiled plasmid contain an RNA polymerase termination signal to avoid rolling circle transcription. Rolling circle transcription will produce a larger than expected RNA transcript when the reaction products are resolved on a denaturing agarose gel. To eliminate this problem, linearizing the template with a restriction enzyme that leaves either a blunt end or a 5′-overhang is preferably effected. Preferably linearizing templates with a restriction enzyme that leaves a 3 ′-overhang is avoided, as RNA polymerases may initiate transcription on the overhang, producing end-to-end transcripts.

It will be appreciated that PCR products can also be used as templates by including an RNA promoter sequence at the 5′ end of either amplification primer. These bases become double-stranded promoter sequences during PCR.

Typically used RNA polymerases are SP6, T7 and T3 polymerases. These RNA polymerases are DNA template-dependent and have distinct, highly specific promoter sequence requirements. Following binding of the of the RNA polymerase to the promoter thereof, the enzyme separates the two DNA strands and uses the 3′>5′ strand as the template for the synthesis of a complementary 5′>3′ RNA strand. Depending on the orientation of the DNA sequence relative to the promoter, the template is designed to generate sense or anti-sense strand RNA.

The DNA template contains a double-stranded promoter region where the polymerase binds and initiates RNA synthesis. Typically used transcription templates are plasmids, which contain two unique RNA polymerase promoters, that flank the multiple cloning site and thus allow transcription of either strand of an inserted sequence.

To enhance mRNA processing, stability and nucleocytoplasmic transport in vivo, the exogenous RNA molecules of the present invention are capped at the 5′ end. Substitution of Cap analogue for a portion of the GTP present in an in vitro transcription reaction will result in the synthesis of transcripts with a cap on the 5′-end of the RNA.

Once generated and preferably purified the exogenous RNA molecule is introduced into the diploid cell. A number of methods for introducing RNA molecules into cells are known in the art. Examples include but are not limited to transfection and microinjection.

A number of transfecting agents for introducing mRNA molecules are known in the art [see Bettinger (2001) Curr. Opin. Mol. Ther. 3:116-124]. Examples include but are not limited to DEAE-dextran [Malone (1989) Proc. Natl. Acad. Sci. USA 86:6077-81], poly(L-lysine) [Fisher (1997) Biochem. J. 321:49-58], dendrimers [Strobel (2000) Gene Ther. 7:2028-35] and DOTAP lipoplexes [Bettinger (2001) Nucleic Acids Res. 29:3882-91].

Preferably, introduction of the exogenous RNA molecule, according to the present invention is effected by microinjection (see Examples section), since this procedure allows cellular introduction of large RNA molecules. Microinjection is the loading or transfer of a dissolved substance (e.g., RNA) into a living cell. Typically, the tip of a glass microcapillary has an inner diameter between 0.2 and 1 μm The capillary is back loaded with the RNA to be transferred into the cells cultured for microinjection.

To visualize and evaluate the success of a microinjection procedure, RNA is typically mixed with dyes or labeled with fluorescent markers such as flourescein or rhodamine. The capillary pricks the cell, and RNA (approximately 10% of the cell volume) is transferred from the capillary into the cell due to pressure exerted on the capillary via the microinjector. Preferred concentration for RNA injection, according to this aspect of the present invention is a volume of about 10% of the cell body at a source concentration of 2-3 μg/μl. Preferred embodiments are described in the Materials and Experimental Procedures section of the Examples section.

As mentioned hereinabove, the diploid cell may be contacted with the agent, prior to, concomitant with or following introduction of the RNA molecule.

As used herein, the term “agent” refers to a molecule or a condition. Examples of molecules which can be utilized as agents according to the present invention include, but are not limited to, nucleic acids, e.g., polynucleotides, ribozymes, and antisense molecules (including without limitation RNA, DNA, RNA/DNA hybrids, peptide nucleic acids, and polynucleotide analogs having altered backbone and/or bass structures or other chemical modifications); proteins, polypeptides, carbohydrates, lipids and “small molecule” drug candidates. “Small molecules” can be, for example, naturally occurring compounds (e.g., compounds derived from plant extracts, microbial broths, and the like) or synthetic organic or organometallic compounds having molecular weights of less than about 10,000 daltons, preferably less than about 5,000 daltons, and most preferably less than about 1,500 daltons.

Examples of conditions suitable for use as agents according to the present invention include, but are not limited to culturing conditions, such as, for example, temperature, humidity, atmospheric pressure, gas concentrations, growth media, contact surfaces, radiation exposure (such as, gamma radiation, UV radiation, X-radiation), injury (e.g., axotomy) and the presence or absence of other cells in a culture.

The agent can be either contacted with or introduced into the cell, using molecular or biochemical methodologies well known in the art. Examples include but are not limited to, transfection, conjugation, electroporation, calcium phosphate-precipitation, direct microinjection, liposome fusion and the like. Selection of a suitable introduction method is dependent upon the host cell and the type of agent used.

Once the exogenous RNA molecule is expressed (i.e., translated), typically between 2-24 hours following introduction, the effect of the agent may be monitored. It will be appreciated that in contrast to DNA, RNA administration directs very rapid expression of the encoded polypeptide thus allowing determination of the studied effect within minutes of RNA administration.

As mentioned hereinabove, monitoring of the phenotype of the diploid cell and/or the expression or activity of the polypeptide within the diploid cell may be effected using fluorescent microscopy. Direct fluorescent microscopy may be applied when the polypeptide includes the detectable portion e.g., GFP, as described above. Alternatively, detection may be effected using fluorescently-labeled antibodies which bind directly or indirectly the polypeptide and/or an epitope tag conjugated thereto.

Alternatively, monitoring is effected by assaying enzymatic activity of the polypeptide or the detectable label, as described above. Examples include but are not limited to kinase activity, phosphatase activity, lipase activity, galacto/glucosidase activity and the like.

Alternatively, the agent may affect the level of expression of the encoded polypeptide. In this case monitoring may be effected using protein expression assays which are well known in the art such as Western blotting and staining.

It will be appreciated that when the normal phenotypic pattern (e.g., level of expression, cellular distribution, biochemical modification, activity etc.) of the polypeptide within the diploid cell is known, such a normal pattern can be used to identify agents which have an effect on the diploid cell and/or on the expression or activity of the polypeptide expressed within the cell.

Alternatively, determination of the effect of the agent on the diploid cell and/or on the expression or activity of the polypeptide expressed within the cell is effected by comparing the pattern (i.e. activity, level and localization) of expression of the polypeptide, following agent treatment, with a similar manipulated cell, which was not treated with the agent.

Still alternatively, the effect of the agent may be determined by comparing the pattern of expression of the polypeptide, prior to, and following agent treatment.

Although the present invention is practiced with a single cell, such a method is preferably used for high throughput screening of agents using a plurality of cells to simultaneously screen a variety of agents. When a large number of cells are microscopically scanned, an automatic high throughput screening is effected using a microscope combined with a digital camera and any one of a number of pattern recognition algorithms, such as the product distributed under the commercial name ARAYSCAN by Cellomics Inc., U.S.A

Thus, in one example, cells are distributed into flat glass-bottom multiwell (96) plates at a precalibrated density that allows the growth of just one or two clones per well. In a typical experiment, between 10-100 plates are prepared and examined microscopically. This screen can be carried out manually. However, it is possible to install an automated stage, for example multiwell attachment for the DeltaVision microscope, Cellomics automated microscope, or an equivalent.

Once identified, agents having an effect on a diploid cell and/or on expression or activity of the polypeptide expressed therewith are preferably recovered.

The retrieved agents are further analyzed for their exact mechanism of action and adjusted for optimal effect, using various biochemical and cell-biology methods. Eventually, distinguishing which of the agent isolated is a potential lead compound can be accomplished by testing the effect of the agent in pharmacological models of various diseases. Agents that affect disease progression or onset, constitute leads for drug development.

In summary, the present invention provides a novel approach for visualizing fate and function of gene expression products within cells, preferably differentiated cells, most preferably neurons. More specifically, the present invention may provide tools to facilitate research on, for example, expression and function of genes; spatiotemporal distribution of gene products; intracellular interactions between genes and gene products; effect of drugs, bioactive materials, neurotransmitters and modulators, electrical activity and manipulation that mimic neurotrauma on gene expression, distribution and function; and analyzing role of transcription factors, membrane properties, signal transduction, growth, regeneration, learning and memory. Hence, the invention provides a useful tool for monitoring expression, distribution and function of genes within cells, that is efficient, sensitive, selective, rapid, convenient, and cost effective.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in. Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Experimental Procedures

Solutions—L-15 supplemented for marine species (msL-15) was prepared as previously described [Schacher and Proshansky (1983)]. Briefly, Leibovitz's L-15 Medium (Gibco-BRL, Paisley, Scotland) was supplemented with 12.5 g/L NaCl, 6.86 g/L D(+) Glucose.H₂O, 3.15 g/L anhydrous MgSO₄, 344 mg/L KCl, 192 mg/L NaHCO₃, 5.7 g/L MgCl₂.6H₂O and 1.49 g/L CaCl₂.2H₂O. Penicillin, streptomycin and amphotericin B (Biological Industries, Kibbutz Beit Haemek, Israel) were added up to final concentrations of 100 units/ml, 0.1 mg/ml and 0.25 μg/ml respectively.

Culture medium included 5-20% filtered hemolymph obtained from Aplysia faciata (specimens were collected along the Mediterranean coast) diluted in ms-L 15.

Artificial Sea Water (ASW) included NaCl 460 mM, KCl 10 mM, CaCl₂ 10 mM, MgCl₂ 55 mM, HEPES 10 mM, adjusted to pH 7.6.

Cell culture—Neurons B1 and B2 from buccal ganglia, MCn neurons from the metacerebral ganglion, sensory neurons from the pleuropeadal ganglion and LUQs from the abdominal ganglion of Aplysia californica were isolated and maintained in culture as previously described [Schacher and Proshansky, (1983); Spira et al., (1993, 1996)].

Briefly, juvenile Aplysia californica (1-10 gr) were anesthetized by injection of isotonic MgCl₂ solution (380 mM) into the animal's body cavity.

Buccal ganglia were dissected and incubated in ms-L15 containing 1% protease (type IX, Sigma, Rehovot, Israel) at 34° C. for 1.5-2.5 h. Following the protease treatment the ganglia were washed with ms-L15, pinned and desheated. The identified neurons were manually pulled out along with their original axon with the aid of a sharp glass microelectrode. The neurons were immediately plated in glass-bottom dishes coated with poly-L-lysine (Sigma, Rehovot, Israel) containing culture medium.

All microinjections were performed 8-24 hours from plating, at room temperature (21-25° C.) after replacing the culture medium with ASW.

Expression constructs—Enzymes were obtained from (New England Biolabs, Beverly, Mass., USA). Each of the chimeric cDNAs of EGFP-EB3, EGFP-p50 and EGFP-MLIV was subcloned into the pCS2+ expression vector as described below [Rupp (1994); Turner and Weintraub (1994)]. For EGFP-EB the pCS2+ vector was cut with XhoI and SnaB. The insert (EGFP-EB3) was cut with NotI, fill in and cut with SalI. For EGFP-P50 the pCS2+ vector was cut with XbaI and the insert was cut with NheI and SpeI.

EGFP cDNA was amplified from pEGFP-N1 vector (Clontech, Palo-Alto, Calif., USA) by polymerase chain reaction (PCR) with two specific primers: 5′-GGCCATGGTGAGCAAGG-3′ and 5′-CTTGTACAGCTCGTCCATG-3′(Genset Oligos) (SEQ ID NOs: 1 and 2, respectively). The PCR product was digested with HindII and SmaI and subcloned into corresponding sites of Bluescript II SK (Stratagen, La Jolla, Calif.). Aplysia actin provided by Dr DesGrosiller (Montreal University, Canada) was amplified by PCR from Bluescript containing the actin cDNA using two specific primers: 5′-ATGTGTGACGACGATGTT-3′ and 5′-TTAGAAGCACTTGCGGTCG-3′ (SEQ ID NOs: 3 and 4, respectively) with SmaI and XbaI restriction sites at their 5′ ends. Following digestion, the PCR product was subcloned in-frame with EGFP into the previously prepared pBluescript-EGFP vector linearized with SmaI and XbaI. The EGFP-Actin fragment was then cut out from pBluescript with ClaI and XbaI and subcloned into corresponding sites of pCS2+ vector [Rupp (1994); Turner and Weintraub (1994) supra].

EYFP, EBFP, ECFP and RFP constructs were prepared as described for EGFP-actin, hereinabove. EYFP, EBFP and ECFP cDNAs were amplified from pEYFP, pEBFP and pECFP, respectively (Clontech) by PCR using the primers set forth in SEQ ID NOs: 1 and 2. The RFP cDNA was amplified from pDsRed1-N1 vector (Clontech) using the following primers: 5′-GGCCACCATGGTGCGCTCCT-3′ and 5′-CAGGAACAGGTGGTGGCGG-3′(Genset Oligos, SEQ ID NOs: 5 and 6, respectively).

Aplysia SNAP-25 provided by Dr W. Sossin, University of Montreal Canada was amplified by PCR using the following SmaI and SpeI restriction sites containing primers: 5′ GTCCCCCGGGATGGCGGCGCCAGCGGAG 3′ and 5′ GCGGACTAGTCTAAGCCTCCTTAAGCAG 3′ (SEQ ID NOs: 7 and 8, respectively). The resultant digested PCR product was used to replace the actin gene from the pCS2-EGFP-actin that was excised by SmaI and XbaI.

PCS2-EGFP-α-tubulin was prepared from Clontech pEGFP-Tub (Cat. #6117-1). This plasmid encodes a fusion protein including EGFP and human α-tubulin. The plasmid was digested with BamHI, filled in with T4 DNA polymerase, digested with NheI and ligated to PCS2 cut by XbaI and SnaBI.

In-vitro transcription—5′-capped and 3′-polyadenylated mRNA was in vitro transcribed using recombinant in vitro transcription system (Promega, Madison, Wis., USA,). 10 μg of NotI-linearized pCS2+ was used as a template to transcribe capped mRNA. The transcription reaction was effected with RiboMax-sp6 kit (Promrga-P1280). Briefly, a reaction mixture was prepared by mixing 8 μl Transcription ×5 Buffer, 8 μl rNTPs mix containing 25 mM CTP, ATP, UTP and 12 mM GTP, 4 μl of 15 mM Cap analog (Roche 85846029), 1 μl of 40 units rRnasin (Promega N251A), 4 μl enzyme mix and 1-2 μg linear plasmid. Final reaction volume was compensated to 40 μl. Reaction was incubated for 2-4 hours in 37° C. RNA was purified by Rneazy mini kit (Qiagene, Cat. No. 74104) and the clean RNA was eluted to a final volume of 25-40 μl and stored at −80° C. The concentration of RNA used for injection did not exceed 5 μg/μl.

mRNA microinjection—mRNA was injected into the cytoplasm of Aplysia neurons bathed in ASW 8-48 hours following plating. 0.5-5 μg/μl mRNA in 80 mM KCl was used for injection. Injection was performed by pressure using Medical System Corp microinjector inserted into the cell body under visual control. Approximately up to 10% of the cell's body volume was injected. The same micropipette used for injection was also used to continuously monitor the transmembrane potential and input resistance [Benbassat and Spira (1993) Exp. Neurol. 122:295-310]. Good penetration was indicated by a resting potential of >−35 mV and measuring a typical input resistance. At the end of the injection, the micropipette tip was pooled out gently of the neuron. Cells were imaged for protein expression 12-48 hours following injection.

Mag-fura-2 Ca²⁺ imaging—To study the local effect of elevated calcium levels on the distribution of a given fluorescentlly labled protein ratio imaging of the free intracellular calcium concentration was effected by mag fura-2. Mag-fura-2 loading, imaging and calibration was done as previously described [Ziv and Spira, (1993, 1995, 1997)]. The fluorescence microscopy system consisted of a Zeiss Axiovert microscope equipped with a 75 W Xenon arc lamp, a Zeiss 40×0.75 NA Plan-Neofluar objective, 340±5 nm and 380±5 nm bandpass excitation filters set in a computer-controlled, Lambda10 position filter changer (Sutter, Novato Calif.), a dichroic mirror with a cut-off threshold of 505 nm and a 545±25 nm band pass emission filter. The images were collected with an intensified CCD video camera (Hamamatsu, Japan), stored as computer files and processed using a software package written in our laboratory.

Proteolytic activity imaging—The effect of activated calpain on the behavior of fluorescentlly labeled protein was imaged on line calpain activity. Imaging of proteolytic activity was performed as previously described [Gitler and Spira, (1998, 2002)]. Neurons which were previously loaded with mag-fura-2 were continuously incubated in ASW containing 10 μM bis(CBZ-Alanyl-Alanine amine) Rhodamine 110 (bCAA-R110, Molecular Probes, Eugene Oreg.) and were imaged for the production of fluorescent Rhodamine 110 (R110). Ratio imaging was used to correct for volumetric changes, and was performed as described for mag-fura-2 except that the excitation wavelengths used were 490±6 nm, which excites R110, and 350±5 nm, which is the isosbestic point of mag-fura-2.

Axotomy—Axonal transection was performed by applying pressure on the axon with the thin shaft of a micropipette under visual control, as previously described [Spira et al., 1993, 1996; Ziv and Spira, (1993)].

### Example 1

### Expression of EGFP and EYFP in Aplysia Neurons

The ability of mRNA injection to direct protein expression in neuronal cells was addressed in cultured Aplysia neurons.

Results

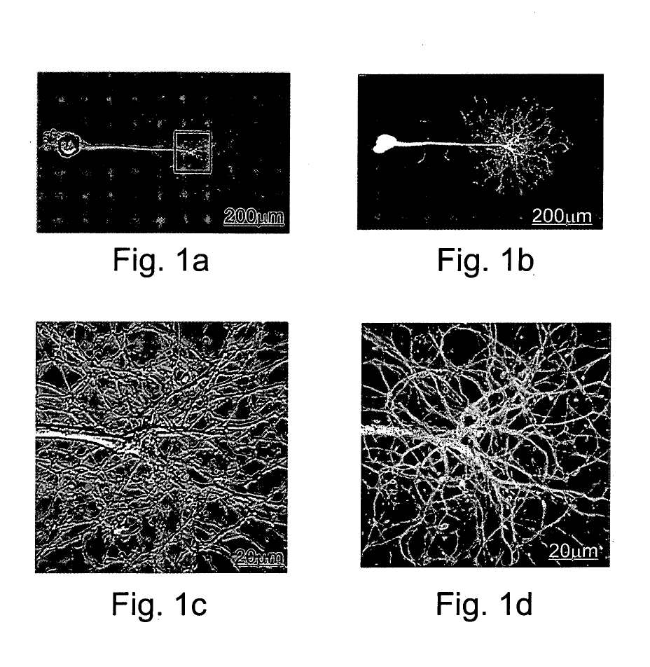

In vitro transcribed mRNA encoding EGFP or EYFP was injected into cultured Aplysia neuron. Cells were microscopically examined 12-24 hours following manipulation. As shown in FIGS. 1a-b, EGFP and EYFP expression was observed in about 100% of the injected neurons. Expression was observed in the cell-body, the axons and the neuritis and the fluorescent signal was evenly distributed in the cytoplasm.

### Example 2

### EGFP-Actin Expression in Cultured Aplysia Neurons

The translational efficiency of mRNAs encoding EGFP-tagged actin and tubulin was examined in cultured Aplysia neurons.

Results

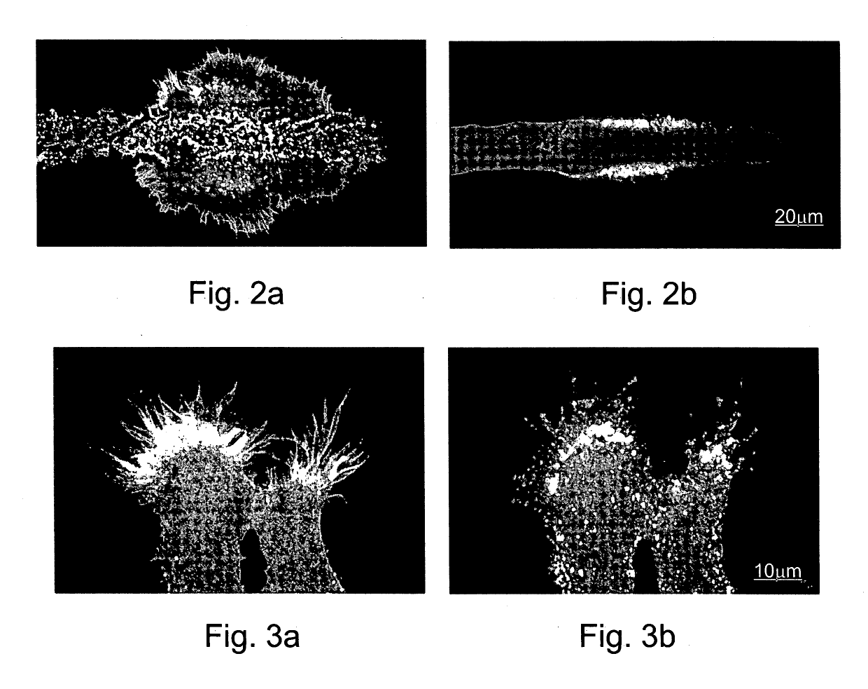

As shown in FIGS. 2a-b, injection of a solution containing mRNA encoding EGFP-actin fusion protein resulted in a high fluorescent signal in the cell body, axons and neuritis. The fluorescent signal appeared to be distributed homogeneously in the cytoplasm of the cell body, main axon and neurites but was not detected within the nucleus. Fluorescent hot spots, possibly representing adhesion plaques, were seen along the plasma membrane facing the substrate.

To determine whether the observed fluorescent signal corresponded to EGFP-tagged actin, rather than to EGFP alone, the main axon was transected and fluorescent signal distribution was imaged during the formation and extension of the lamellipodium of the growth cone. As previously described, axonal transection of cultured Aplysia neuron leads to the rapid formation of a growth cone lamellipodium at the tip of the cut axon [Ziv and Spira, (1995); Ashery et al., (1996); Gitler and Spira, (1998, 2002); Spira et al., (2001)]. It is also well documented that the growth cone's lamellipodium perimeters are rich with actin filaments that are central to its motility (Forscher and Smith, 1988; Lin et al., 1994; Tanaka and Sabry, 1995; Scheafer et al., 2002).

Indeed as shown in FIGS. 2a-b, following axotomy of manipulated neurons, a growth cone in the form of large lamellipodium was evident within 10_{—}/15 min of axotomy. The perimeters of the growth cone were rich with fluorescent actin bundles while the central region exhibited fluorescent puncta (FIGS. 2a-b). As shown in FIGS. 3a-b, the fluorescent bundles depolymerised within minutes of cytochalasin B application.

Thus, it is concluded that these bundles were EGFP labelled actin bundles. These results demonstrate that the fluorescent signal corresponds to expressed EGFP-actin fusion protein, which incorporates into the actin skeleton and allows the visualisation of the dynamic behaviour of this skeletal component.

### Example 3

### Expression of EGFP-Tagged Tubulin in Cultured Aplysia Neurons

To establish that the above-described methodology can be used as a reliable tool to express various types of proteins in cultured Aplysia neurons, the mRNA of EGFP-tagged tubulin was injected into cultured Aplysia neurons.

Results

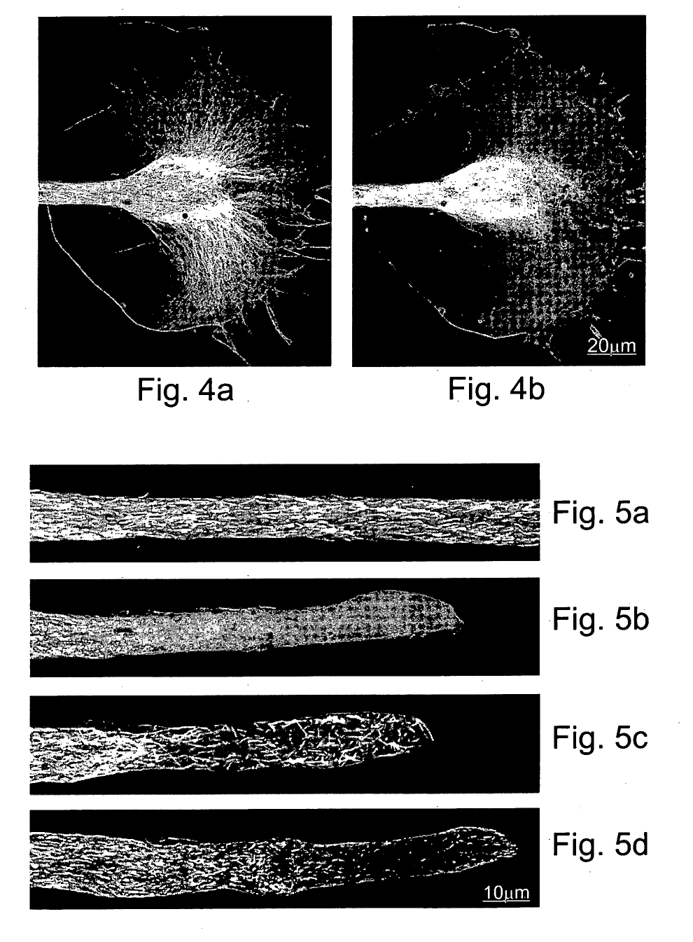

On-line confocal microscope imaging of neurons injected by a solution containing mRNA encoding EGFP-a tubulin fusion protein resulted in incorporation of the tagged tubulin into microtubules that extended into an axotomy induced growth cone's lamellipodium (FIG. 4a). Bath application of the microtubules depolymerizing agent nocodazole (5 mM) for 5 min resulted in depolymerisation of most microtubules (FIG. 4b).

### Example 4

### Expression of EYFP-Tagged SNAP-25 in Cultured Aplysia Neurons

Similarly to Example 3, above, SNAP-25 detectable protein was injected into cultured Aplysia neuroms. SNAP-25 is a member of the SNARE complex, the synaptosome associated protein of 25 kDa (SNAP-25).

The fluorescent signal of SNAP-25 revealed the presence of fluorescent spots in the intact axon as well as in the growth cone (not shown).

### Example 5

### Expression of the End Binding Protein 3 (EB3) in Aplysia Neurons

Background

End Binding Protein 1 (EB1), is a protein known to bind to APC (adenomatus polyposis coli tumor suppressor gene) which depletes cytoplamic β-catenins. EB1 associate with MTs of the mitotic spindle and is important in spindle assembly throughout the cell cycle. End Binding Protein 3 (EB3) a homologue of EB1, was recently isolated from human fetal brain [Nakagawa et al., (2000)]. The full-length cDNA of EB3 encodes a protein of 282 amino acids with 54% identity to EB1 but is expressed preferentially in brain tissue. EB3 binds to APCL which is thought to play a role in differentiation of the nervous system. The conservation of the armadillo domain by APC and APCL (76% identity) suggests that both interact with similar proteins. APCL can interact with β-catenin, and deplete intracellular β-catenin as APC does.

To shed light on the function of EB3 in neuronal cells, a GFP-EB3 mRNA was injected into cultured Aplysia neurons and protein expression, localization and activity was determined.

Results

As shown in FIG. 5a, GFP-EB3 was expressed within 5 hr of mRNA injection into cultured Aplysia neurons. GFP-EB3 was able to bind in stretches to the plus end of microtubules, move with the growing MT's tips to thereby form a comet tail-like structure.

Thus, the expression of human GFP-EB3, in cultured Aplysia neurons enabled to probe on line the polarity and dynamics of MTs in the neurons, the dynamic of microtubules organizing center, the role of MTs in GCs formation, neurites extension and the effect of drugs on MTs.

### Example 6

### Imaging cellular Cascades Using EGFP-EB3-Expressing Aplysia Neurons

Background

To illustrate the utility of cultured Aplysia neurons as an expression platform for mammalian genes, several parameters which are involved in the cellular cascade leading to the formation of a growth cone following axotomy were imaged. Such a cascade is initiated by a transient and local elevation of the free intracellular Ca²⁺ concentration ([Ca²⁺]ᵢ), which is followed by localized activation of the calcium dependent protease calpain. Calpairi-dependent proteolysis leads to restructuring of the microtubules (MTs) and neurofilaments to form a specialized cytoskeletal compartment that “traps” and “hold” Golgi derived transported vesicles. The “vesicles trap” is formed within seconds of axotomy 100-150 μm posterior to the site of transection.

Results

Confocal microscope imaging of EGFP-EB3 throughout the process revealed that during the elevation of the [Ca²⁺]ᵢ the MTs undergo two cycles of depolymerizations (FIG. 5b) and repolymerizations (FIG. 5c). Thereafter, the MTs repolymerize to form the vesicles “trap” by the reorientation of MTs plus ends into a common center (FIG. 5d).

Local elevation of the [Ca²⁺]ᵢ by ionomycin application was able to mimic the process, demonstrating that the transient elevation in the [Ca²⁺]ᵢ rather than other injury related events underlie the process. In addition to that it was also observed that inhibition of calpain by calpeptin induces milled dissociation of EB3 from the MTs. Nacodasol induces MTs deplymerization and dissociation of EB3 from the MTs.

### Example 7

### Expression of EGFP-Dynamitin (P50) Construct

Through interactions with organelle-bound MT motors translocate organelles. The Kinesin family motors translocate organelles toward the plus end (cell periphery) and dynein translocate toward the minus end (cell center). Dynactin is a multisubunit complex that plays an accessory role in cytoplasmic dynein function. P50 (GenBank Accession No. AF200744), is a subunit of the Dynactin complex. Focusing of the minus ends into radial array is generally related to MTs outgrowth from the centrosom. Nevertheless, dynein forms complexes that are capable of interacting with more than one MT. Since under these conditions dynein remains attached to the minus end (in centrosom free conditions and the presences of active dynein) a self-organization condition is formed which drives the MTs to form a focused array. GFP-p50 was expressed within 5 hr. of mRNA injection into cultured Aplysia neurons.

### Example 8

### Expression of ML VIV (Mucolipidosis Type IV)

Mucolipidosis type VI is a neurodegenerative lysosomal storage disorder characterized by psychomotor retardation. The diseases is classified as mucolipidosys due to the simultaneous lysisosomal storage of lipids and water soluble substrate. The MLVIV gene is involved in regulation of the endocytotic pathway (Bach 2001). GFP-MLVIV was expressed within 5 hr. of mRNA injection into cultured Aplysia neurons.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### Additional References Are Cited In The Text

Ashery. U., Penner R., and M. E., Spira. (1996) Acceleration of memebrane recycling by axotomy of cultured Aplysia neurons. Neuron 16:641-51.

Benbassat D. and M. E., Spira (1993) Survival of isolated axonal segment in culture: Morphological, ultrastructural and physiological analysis. Exp. Neurol. 122:295-310.

Chang. D. J., Li. X. C., Lee. Y. S., Kim H. K., Kim. U. S., Cho, N. J., Lo, X., Weiss, K R., Kandel, E. R., and Kaang B. K. (2000). Activation of heterologusly expressed octopamine receptor coupled only to adenylyl cyclase produces all the features of presynaptic facilitation in Aplysia sensory neurons. Proc. Nat. Acad. Scie. USA, 97: (4) 1829-1839.

Coquelle, F. M., Caspi, M. Cordellieres F. P., Dompierre J. P., Dujardin D. L., Koifman, C., Martin, P., Hoogenraad, C. C., Akhmanov, A., Galjart, N., DeMay, J. R., Reiner, O. (2002). LISI, CLIP-170's key to the dynein/dynactin pathway. Molecular and cellular biology 22: (9) 3089-3102.

DesGroseillers, L., cowan, D., Miles, M., aweet, A. And Scheller, R. H. (1987) Aplysia californica neurons express microinjected neuropeptide gene. Mol. Cell. Biol. 7:2762-2771.

Forscher P, Smith S J. 1988. Actions of cytochalasins on the organization of actin filaments and microtubules in a neuronal growth cone. J. Cell Biol 107:1505-1516.

Gabso M, Neher, E., Spira M. E. 1997. Low mobility of the Ca²⁺ buffers in axons of cultured Aplysia neurons. Neuron 18:473-48 1.

Gitler, D., Spira, M. E. (1998). Real time imaging of calcium-induced localized proteolytic activity after axotomy and its relation to growth cone formation. Neuron 20:1123-1135.

Gitler D, Spira M E. 2002. A short window of opportunity for calpain induced growth cone formation after axotomy of Aplysia neurons. J Neurobiol 52:267-279.

Kaang B. K. (1996). Parameters influencing ectopic gene expression in Aplysia neurons. Neuroscience Letters 221: 29-32

Kaang B. K. (1996). Neuronal expression of reporter genes in the intact nervous system of Aplysia. Mol. cells, 6: 285-295.

Kaang B. K., Kandel, E. R. and Grant, S. G. N., (1993). Activation of cAMP-responsive genes by stimuli that produce long term facilitation in Aplysia sensory neurons. Neuron, 10: 427-435.

Kaang B. K., Pfaffinger, P. J., Grant, S. G. N., Kandel, E. R. and Furakawa, Y. (1992). Over expression of an Aplysia shaker K+ channel gene modifies the electrical properties and synaptic efficacy of identified Aplysia neuron. Proc. Nat. Acad. Scie. USA, 89: 1133-1137.

Kandel, E. R. (2001) The molecular biology of memory storage: A dialog between genes and synapses Science 294: 1030-1038.

Kim, H. K. Kaang, B. K. (1998) Truncated green fluorescent protein mutant and their expression in Aplysia neurons. Brain research bulletin 47,(1) 35-41.

Lee, Y. S., Lee, J. A., Jung, J., Oh, U., Kaang, B. K. (2000). The cAMP-dependent kinase pathway dose not sensitize the cloned vanilloid receptor type 1 expressed in Xenopus oocytes or Aplysia neurons. Neuroscience letters 288: 57-60.

Lee et al. (2001) Learn Mem. 8:220-6.

Lin C H, Forscher P. 1993. Cytoskeletal remodeling during growth cone-target interactions. J Cell Biol 121:1369-1383.

Lin C H, Thompson C A, Forscher P. 1994. Cytoskeletal reorganization underlying growth cone motility. Curr Opin Neurobiol 4:640-647.

Martin, K. C., Hu, Y., Armitage, B. A. Seigelbaum S. A., Kandel, E. R., Kaang B. K, (1995). Evidence for synaptotagmin as an inhibitory clamp on synaptic vesicles release in Aplysia neurons. Proc. Nat. Acad. Scie. USA, 92: 11307-11311.

Manseau (2001) J. Neurosci. 21:1247-56.

Misawa (2000) PNAS 97:3062-3066

Mochida, S., Poulin, B., Eisel, U., Binz, T., Kurazono, H., Neimann H., Tauc, L.(1990). Exogenous mRNA encoding tetanus or botulinum neurotoxins axpressed in Aplysia neurons. Proc. Nat. Acad. Scie. USA, 87: 7844-7848.

Nakagawa, H., Koyama K., Murata, Y., Akiyama T. and Y. Nakamura (2000). EB3, a novel member of the EB1 family preferentially expressed in the central nervous system, binds a CNS-specific APC homologue. Oncogen 19: 210-216

Rupp et al. (1994) Genes Dev. 8:1311-23.

Schacher S, Proshansky E. 1983. Neurite regeneration by Aplysia neurons in dissociated cell culture: modulation by Aplysia hemolymph and the presence of the initial axonal segment. J Neurosci 3:2403-2413.

Scheafer et al., (2002) J. Cell Biol. 158(1):139-52.

Spira M E, Benbassat D, Dormann A. 1993. Resealing of the proximal and distal cut ends of transected axons: electrophysiological and ultrastructural analysis. J Neurobiol 24:300-316.

Spira M E, Dormann A, Ashery U, Gabso M, Gitler D, Benbassat D, Oren R, Ziv N E. 1996. Use of Aplysia neurons for the study of cellular alterations and the resealing of transected axons in vitro. J Neurosci Methods 69:91-102.

Spira M E, Gitler D, Oren R, Ziv N E. 1999. Calcium homeostasis and mechanisms of growth cone formation during neuritogenesis in cultured Aplysia neurons. In: Haynes L W, editor. The neuron in tissue culture. Chichester: John Wiley and Sons Ltd., p 87-103.

Spira M E, Ziv N E, Oren R, Dormann A, Gitler D. 2000. High calcium concentrations, calpain activation and cytoskeleton remodeling in neuronal regeneration after axotomy. In: Pochet R, Donato R, Haiech J, Heizmann C W, Gerke V, editors. Calcium: The molecular basis of calcium action in biology and medicine. Dordrecht/Boston/London: Kluwer Academic Publishers, p 581-597.

Spira et al. (2001) Cell Mol. Neurobiol. 21(6):8:591-03.

Spira et aL (2003) J Comp. Neurol. 453:293-319.

Suter D M, Forscher P. 2000. Substrate-cytoskeletal coupling as a mechanism for the regulation of growth cone motility and guidance. J Neurobiol 44:97-113.

Tanaka (1995) Cell 83:171-6.

Turner and Weintraub (1994) Genes Dev. 8: 1434-47.

Zhao, B. Rassendren, F., Kaang, B. k. Furukauz, Y., Kubo, T., and Kandel E. R. (1994). A new class of non inactivating channels from Aplysia capable of contributing to the resting potential and firing pattern of neurons. Neuron 13: 1205-1213

Ziv N E, Spira M E. 1993. Spatiotemporal distribution of Ca²⁺ following axotomy and throughout the recovery process of cultured Aplysia neurons. Eur J Neurosci 5:657-668.

Ziv N E, Spira M E. 1995. Axotomy induces a transient and localized elevation of the free intracellular calcium concentration to the millimolar range. J Neurophysiol 74:2625-2637.

Ziv N E, Spira M E. 1997. Localized and transient elevations of intracellular Ca²⁺ induce the dedifferentiation of axonal segments into growth cones. J Neurosci 17:3568-3579.

Ziv N E, Spira M E. 1998. Induction of growth cone formation by transient and localized increases of intracellular proteolytic activity. J Cell Biol 140:223-232.

What is claimed is:

1. A method of determining the effect of an agent on a diploid cell and/or on an expression or activity of a polypeptide expressed within the diploid cell, the method comprising:
(a) administering an exogenous RNA molecule encoding the polypeptide into the diploid cell;
(b) contacting the diploid cell with the agent; and
(c) monitoring a phenotype of the diploid cell and/or the expression or activity of the polypeptide within the diploid cell, thereby determining the effect of the agent on the diploid cell and/or on the expression or activity of the polypeptide expressed within the diploid cell.

2. The method of claim 1, wherein the diploid cell is a differentiated cell.

3. The method of claim 2, wherein the diploid cell is a neuron.

4. The method of claim 1, wherein said administering is effected by microinjection.

5. The method of claim 1, wherein said exogenous RNA molecule is a capped messenger RNA.

6. The method of claim 1, wherein the polypeptide is conjugated to a detectable label selected from the group consisting of green fluorescent protein (GFP), derivatives of GFP, luciferase, β-glucoronidase, β-galactosidase, and chloramphenicol acetyltransferase.

7. The method of claim 1, wherein said monitoring is effected by:
(i) fluorescent microscopy;
(ii) protein expression assay; and/or
(iii) assaying enzymatic activity.

8. The method of claim 1, wherein said exogenous RNA molecule encoding the polypeptide is a chimeric RNA molecule including a first sequence region encoding the polypeptide and a second sequence region encoding a reporter molecule, wherein said first and said second sequence regions are linked via an internal ribosome entry site sequence.

9. The method of claim 1, wherein said exogenous RNA molecule encoding the polypeptide is a chimeric RNA molecule including a first sequence region encoding the polypeptide and a second sequence region encoding a reporter molecule, wherein said first and said second sequence regions are in-frame linked.

10. A neuronal cell comprising a chimeric RNA molecule including a first sequence region encoding a polypeptide of interest and a second sequence region encoding a reporter molecule, wherein said first and said second sequence regions are linked via an internal ribosome entry site sequence.


SEQUENCE LISTING (8 sequences)

SEQ ID NO: 1 - DNA, 17 bp, Artificial sequence
  Single strand DNA oligonucleotide
ggccatggtgagcaagg

SEQ ID NO: 2 - DNA, 19 bp, Artificial sequence
  Single strand DNA oligonucleotide
cttgtacagctcgtccatg

SEQ ID NO: 3 - DNA, 18 bp, Artificial sequence
  Single strand DNA oligonucleotide
atgtgtgacgacgatgtt

SEQ ID NO: 4 - DNA, 19 bp, Artificial sequence
  Single strand DNA oligonucleotide
ttagaagcacttgcggtcg

SEQ ID NO: 5 - DNA, 20 bp, Artificial sequence
  Single strand DNA oligonucleotide
ggccaccatggtgcgctcct

SEQ ID NO: 6 - DNA, 19 bp, Artificial sequence
  Single strand DNA oligonucleotide
caggaacaggtggtggcgg

SEQ ID NO: 7 - DNA, 28 bp, Artificial sequence
  Single strand DNA oligonucleotide
gtcccccgggatggcggcgccagcggag

SEQ ID NO: 8 - DNA, 28 bp, Artificial sequence
  Single strand DNA oligonucleotide
gcggactagtctaagcctccttaagcag